(19) United States
(12) Patent Application Publication
(10) Pub. No.: US 2002/0172647 A1
(43) Pub. Date: Nov. 21, 2002

(54) Composition containing a steroid and a liposoluble UV filter

(75) Inventor: Carole Guiramand, Jouy En Josas (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/091,317

(22) Filed: Mar. 6, 2002

(51) Int. Cl. A61K 31/57; A61K 7/42; A61K 31/56

(52) U.S. Cl. 424/59; 514/171

(57) ABSTRACT

This invention relates to a composition containing, at least one DHEA compound and a liposoluble UV filter. The liposoluble UV filter is believed to make it possible to prevent or delay crystallization of the DHEA compound. The invention further relates to cosmetic and dermatological uses of this composition, in particular to prevent or treat chronological or actinic cutaneous aging.

### REFERANCE TO RELATED APPLICATION

This application claims priority to provisional application Serial No. 60/273,341, incorporated herein by reference.

### BACKGROUND OF THE INVENTION

1. Field of the Invention

This invention relates to a composition containing, preferably in a physiologically acceptable medium, DHEA and/or a metabolic/biological precursor thereof and/or a metabolic/biological derivative thereof, characterized in that it also includes at least one liposoluble UV filter. The invention also relates to the uses of this composition, and to a process for solubilization of the aforementioned DHEA materials by at least one liposoluble UV filter.

2. Discussion of the Background

DHEA, or dehydroepiandrosterone, is a natural steroid produced essentially by the adrenocortico glands. It is known for its capacity to promote keratinization of the epidermis (JP-07 196 467), or even in the treatment of dry skin, by reason of its ability to increase endogenous production and secretion of sebum and to strengthen the barrier effect of the skin (U.S. Pat. No. 4,496,556). The use of DHEA to remedy dermal atrophy through inhibition of the loss of collagen and of connective tissue also has been described in the U.S. Pat. No. 5,843,932. Combinations of DHEA and retinoids have been described in U.S. Pat. No. 6,093,706 as useful to treat oxidative epithelial damage. Finally, it has been proposed to use DHEA sulfate to treat various signs of aging such as wrinkles, the loss of skin radiance and cutaneous slackening (EP-0 723 775). Thus the advantage there may be in using DHEA in cosmetic or dermatological compositions is understood.

Now, DHEA is solubilized only with difficulty in aqueous and hydroalcoholic media, which limits its formulation in cosmetic and dermatological compositions administered topically or orally. It likewise has a tendency to recrystallize or to deteriorate. There ensues a more or less significant loss of efficacy of these compositions, depending on the degree of recrystallization and/or deterioration, which defeats the sought objective. In addition, this recrystallization or deterioration can alter the overall stability of these compositions as well as their appearance, which can turn the user away from them.

Thus there is a need to solubilize DHEA, as well as its precursors and/or derivatives posing the same problems of recrystallization, preferably in a physiologically acceptable solubilizing agent.

### SUMMARY OF THE INVENTION

The inventors have now discovered, in surprising manner, that these DHEA compounds can be solubilized easily with liposoluble UV filters.

### OBJECTS OF THE INVENTION

The invention, therefore, has as one of its object a composition containing, preferably in a physiologically acceptable medium, DHEA and/or a metabolic/biological precursor/derivative thereof (DHEA compounds), characterized in that it also includes at least one liposoluble UV filter, with the exclusion of para-aminobenzoic acid, methyl cinnamate, 4-methoxy octyl cinnamate, titanium oxide and zinc oxide.

It further has as its object a process for solubilization of the DHEA compounds including mixing one or more of said DHEA compounds with at least one liposoluble UV filter, with the exclusion of the previously cited filters. Mixing may be implemented cold, at room temperature, or heated, for example to 75° C., generally under agitation.

### DETAILED DESCRIPTION OF THE INVENTTON

DHEA has the following formula (I):

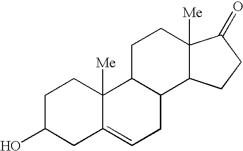

DHEA which may be used according to the invention is, for example, available from the company AKZO NOBEL.

Biological precursors of DHEA include in particular Δ5-pregnenolone and 17α-hydroxy pregnenolone, this list not being limitative. Metabolic derivatives of DHEA include in particular Δ5-androstene-3,17-diol and Δ4-androstene-3,17-dione, as well as 7αOH-DHEA, 7βOH-DHEA, those forms of DHEA described in U.S. 6,093,706, and 7-keto-DHEA, this list not being limitative. 7αOH-DHEA is preferred for a use in this invention. These precursors and derivatives can either be purchased commercially or prepared by one of ordinary skill in the art. As noted above, DHEA and metabolic/biological precursors and derivatives of DHEA are referred to herein as “DHEA compounds” and “DHEA compound.” Preferred DHEA compounds are DHEA and the specific precursors and derivatives listed supra. Another preferred group of DHEA compounds are DHEA and those precursors and derivatives that pose recrystallization and/or deterioration problems in solution. One or more DHEA compounds may be present in the invention compositions.

The composition according to this invention contains at least one (i.e., one or two or more) liposoluble UV filter, which is believed to act as a solubilizing agent for the above-mentioned DHEA compounds.

Examples of liposoluble UV filters include:

(1) derivatives of salicylic acid such as homomenthyl salicylate, 2-ethylhexyl salicylate, triethanolamine salicylate, 4-isopropylbenzyl salicylate;

(2) derivatives of cinnamic acid other than methyl cinnamate and 4-methoxy octyl cinnamate such as 4-methoxy isopentyl cinnamate, 4-methoxy isoamyl cinnamate, 4-methoxy diethanolamine cinnamate;

(3) liquid derivatives of β,β′-diphenylacrylate;

(4) derivatives of substituted para-aminobenzoic acid such as 2-ethylhexyl p-dimethylaminobenzoate and glycerol p-aminobenzoate;

(5) derivatives of dibenzoylmethane;

(6) liposoluble derivatives of benzophenone;

(7) benzotriazole silicones, in particular those described in patent application EP-A-0 392 883;

(8) the silicic derivatives of N-substituted benzimidazolyl-benzazoles or of benzofuranyl-benzazoles, in particular those described in patent application EP-1 028 120; and

(9) mixtures thereof.

Among the salicylic acid derivatives which may be used according to the invention, preferred materials include:

homomenthyl salicylate (also known under the name of homosalate), sold in particular under the designation “KEMESTER HMS” by the Witco Company. It corresponds to the following formula:

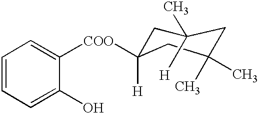

octyl salicylate sold in particular under the commercial designation “UVINUL O-18” by the BASF Company, and it corresponds to the following formula:

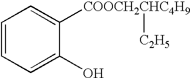

The liquid β,β′-diphenylacrylate derivatives which may be used according to this invention preferably are chosen from among those corresponding to the following formula (V):

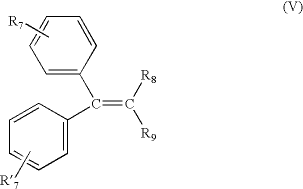

in which:

R₇ and R′₇, which may be the same or different, are in a meta or para position and are chosen from among: hydrogen; a linear or branched chain C₁-C₈ alkoxy radical; a linear or branched chain C₁-C₄ alkyl radical;

R₈ represents a radical chosen from among the radicals —CO—O—R₈₁, —OR₈₁, —CO—NR₈₁, and —CO—R₈₁, in which R₈₁ represents a linear or branched chain C₂-C₂₀ alkyl radical (R₈₁, preferably represents a linear or branched chain C₄-C₈ alkyl radical);

R₉ represents a hydrogen atom or a radical chosen from among the radicals —CN, —NH₂, —OH, —OR₉₁, —R₉₂—OH, —CO—NH₂, and —CH(OR₉₃)—CH₂—OR₉₃, in which:

R₉₁ represents a sugar moiety, or an ethylene polyoxide,

R₉₂ represents a linear or branched chain C₁-C₃ alkylene radical,

R₉₃ represents a hydrogen atom or a —CO—CH₃ radical.

According to an advantageous aspect of the invention, the liposoluble UV filters are chosen from among the β-β′-diphenylacrylates.

Among the liquid β,β′-diphenylacrylate derivatives of formula (V) which may be used according to this invention, most particularly preferred is 2-ethylhexyl α-cyano-β,β′diphenylacrylate, also called octocrylene, and known for being a lipophilic UVB-absorbent filter. It is available commercially and sold in particular under the name of “UVINUL N 539” by the BASF Company. It corresponds to the following formula:

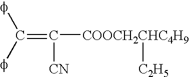

in which ø designates a phenyl radical.

Among the derivatives of p-aminobenzoic acid, especially preferred is 2-ethylhexyl para-dimethylaminobenzoate sold in particular under the designation “ESCALOL 507” by the ISP Company.

As derivatives of dibenzoylmethane, it is especially preferred to use 4-(tert-butyl) 4′-methoxy dibenzolymethane, in particular that offered for sale under the commercial designation of “PARSOL 1789” by the GIVAUDAN Company.

Among the liposoluble derivatives of benzophenone, there may be cited 2-hydroxy-4-methoxy-benzophenone, also called oxybenzone (benzophenone-3) such as the product sold under the name UVINUL M40 by the company BASF.

As benzotriazole silicones, it is preferred to use the compound of the following formula, or 2-(2H-benzotriazole-2-yl)-4-methyl-6-[2-methyl-3-[1,3,3,3-tetramethyl-1-[(trimethylsilyl)oxy] disiloxanyl]propynl]phenol:

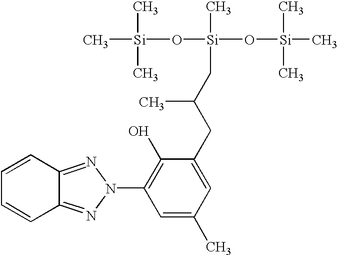

The silicic derivatives of N-substituted Benzimidazolyl-Benzazoles or of Benzofuranyl-Benzazoles which are especially well suited for a use in this invention are in particular:

2-[1-[3-[1,3,3,3-tetramethyl-1-[(trimethylsilyl)oxy]disiloxanyl]propyl]-1H-benzimidazol-2-yl]-benzoxazole

2-[1-[3-[1,3,3,3-tetramethyl-1-[(trimethylsilyl)oxy]disiloxanyl]propyl]-1H-benzimidazol-2-yl]benzothiazole

2-[1-(3-trimethylsilanyl-propyl)-1H-benzimidazol-2-yl]benzoxazole

6-methoxy-1,1′-bis-(3-trimethylsilanyl-propyl)-1H, 1′H-[2,2′]bibenzimidazolyl-benzoxazole

2-[1-(3-trimethylsilanyl-propyl)-1H-benzimidazol-2-yl]-benzothiazole.

The concentration of DHEA compound(s) in the composition according to the invention advantageously ranges between 0.001% and 20% by weight, preferably between 0.01 and 10% by weight, most preferably between 0.1 and 3% by weight, with respect to the total weight of the composition including 0.5, 1, 2, 4, 5, 6, 7, 8 and 9% by weight and all values and subranges between stated values. In addition, the quantity by weight of liposoluble UV filter advantageously represents from 0.1% to 20%, better, from 0.5 to 10%, of the total weight of the composition, including all values and subranges therebetween such as 1, 2, 3, 4, 5, 6, 7, 8, 9, 11, 13, 15, 17 and 19% by weight based on total weight.

The composition according to the invention may be in any form, and is not limited. For example, the invention composition may exist in all the galenical forms used for topical application on the skin, in particular in the form of an oily solution, an oil-in-water or water-in-oil or mixed or multiple emulsion, a silicone emulsion, a microemulsion or nanoemulsion, an oily gel or an anhydrous liquid, pasty or solid product, etc.

The invention composition may be more or less fluid and can have the appearance of a white or tinted cream, a pomade, a milk, a lotion, a serum, a paste, a mousse or a gel. It may be applied on the skin in spray form. It also may exist in solid form, for example in stick form. It may be used as a care product and/or as a makeup product for the skin, or as a hair product, for example as a shampoo or conditioner.

The composition of the invention also may contain additives customary in the cosmetic and dermatological areas, such as hydrophilic or lipophilic gelling agents, hydrophilic or lipophilic active substances, preservatives, antioxidants, solvents, fragrances, fillers, pigments, odor absorbers and coloring agents. The quantities of these various additives are those traditionally used in the areas under consideration, for example from 0,01 to 20% of the total weight of the composition. Depending on their nature, these additives may be introduced in the fatty phase, in the aqueous phase, in lipid vesicles and/or in nanoparticles. These additives, as well as their concentrations, should be such that they are not detrimental to the advantageous properties of the DHEA compounds according to the invention.

Also, and for example, depending on the Solar Protection Factor which one wishes to impart to the composition according to the invention, it advantageously may contain one or more hydrosoluble UV filters. Examples of hydrosoluble UV filters include:

(1) the filters derived from benzylidene camphor, an especially preferred example of which is benzene 1,4-[di(3-methylidenecampho 10-sulfonic)] acid described in particular in patent applications FR-A-2 528 420 and FR-A-2 639 347;

(2) the filters of the benzimidazole or benzoxazole type, such as 2-phenyl benzimidazole 5-sulfonic acid, available from the company MERCK under the commercial designation EUSOLEX 232;

(3) the hydrosoluble derivatives of benzophenone, such as 2-hydroxy-4-methoxy-benzophenone-5-sulfonic acid, as well as its sodium sulfate form (benzophenone-5) sold under the name UVINUL MS 40 by the company BASF; and

(4) mixtures thereof.

When the composition according to the invention is an emulsion, the proportion of the fatty phase is not limited and may preferably range from 5 to 80% by weight, and more preferably from 5 to 50% by weight with respect to the total weight of the composition. Fatty substances, emulsifiers and coemulsifiers used in the composition in the form of emulsion may be chosen from among those traditionally used in the area under consideration. The emulsifier and the coemulsifier (when used) preferably are present in the composition in a proportion ranging from 0.3 to 30% by weight, and preferably from 0.5 to 20% by weight with respect to the total weight of the composition.

Examples of fatty substances usable in the invention include solubilizing agents with a base of 2-alkyl alkanols and the esters thereof, oils and in particular mineral oils (vaseline oil), oils of vegetable origin (avocado oil, soy oil), oils of animal origin (lanolin), synthetic oils (perhydrosqualene), silicone oils (cyclomethicone) and fluorous oils (perfluoropolyethers). Fatty alcohols such as cetyl alcohol, fatty acids, waxes and rubbers and in particular silicone rubbers, also may be used as fatty substances.

Emulsifiers and coemulsifiers which may be used in the invention include, for example, fatty acid and polyethylene glycol esters such as PEG-100 stearate, PEG-50 stearate and PEG-40 stearate; fatty acid and polyol esters such as glyceryl stearate, sorbitan tristearate and the oxyethylene sorbitan stearates available under the commercial designations Tween° 20 or Tween® 60, for example; and mixtures thereof.

Examples of hydrophilic gelling agents include in particular the carboxyvinyl polymers (carbomer), the acrylic copolymers such as the acrylate/alkylacrylate copolymers, the polyacrylamides, the polysaccharides, the natural rubbers and the clays; and, as lipophilic gelling agents, examples include the altered clays such as bentones, the metal salts of fatty acids and hydrophobic silica.

The composition according to the invention has a preferred application in the prevention and treatment of signs of chronological and actinic aging, as well as in the treatment of certain pathologies. This invention, therefore, also relates to the cosmetic use of the composition indicated above for preventing or treating the signs of chronological or actinic cutaneous aging, in particular wrinkles and lines and/or cutaneous slackening. Most particularly, the composition according to the invention is intended for the prevention or treatment of actinic aging.

This invention also relates to the use of the composition described above for manufacturing a preparation intended to prevent or treat the signs of chronological or actinic cutaneous aging.

In any case, the composition according to the invention and/or the preparation obtained therefrom preferably contains an effective quantity of DHEA compound(s), sufficient for producing the sought result, and a physiologically acceptable medium.

The invention will be illustrated by the following non-limitative examples. In these examples, the quantities are indicated in weight percentage, unless otherwise noted.

### EXAMPLES

### Example 1

### Demonstration of the Solubilizing Effect of the Liposoluble UV Filters

Three compositions C1 and C2 are prepared as defined in Table 1.

TABLE 1

| | C1 | C2 |
|---|---|---|
| Polyglycerol distearate (2 mol) | 2% | 2% |
| PEG mono-stearate (8 OE) | 1.35% | 1.35% |
| Stearic acid | 1% | 1% |
| Preservatives | 1.35% | 1.35% |
| DHEA | 1% | 1% |
| Octocrylene | 5% | 0% |
| C₁₂₋₁₅ alcohol benzoate | 20% | 25% |
| Neutralizers | 0.45% | 0.45% |
| Propylene glycol | 10% | 10% |
| Gelling agent | 0.5% | 0.5% |
| Water | qsp 100% | qsp 100% |

After fifteen days at 4° C. or at room temperature, the Cl composition does not have any DHEA crystals, while the C2 composition already has DHEA crystals, visible through the microscope under polarized light, at the end of 24 hours of storage at 4° C. or at room temperature.

It therefore is clearly apparent from Table 1 that the liposoluble UV filters make it possible to solubilize DHEA compounds.

### Example 2

### Cosmetic Composition

| | |
|---|---|
| Butyl methoxy dibenzoylmethane | 5% |
| 7αOH-DHEA | 1% |
| Polyglycerol distearate (2 mol) | 2% |
| PEG mono-stearate (8 OE) | 1.35% |
| Stearic acid | 1% |
| Propylene glycol | 10% |
| Preservative | 1.35% |
| Gelling agent | 0.5% |
| Neutralizers | 0.45% |
| Water | qsp 100% |

The above composition may be used as a care cream intended to combat wrinkles and to prevent the loss of skin firmness and elasticity.

### Example 3

### Cosmetic Composition

| | |
|---|---|
| Octyl salicylate | 5% |
| 7-keto-DHEA | 1% |
| polyglycerol distearate (2 mol) | 2% |
| PEG mono-stearate (8 OE) | 1.35% |
| Stearic acid | 1% |
| Propylene glycol | 10% |
| Preservative | 1.35% |
| Gelling agent | 0.5% |
| Neutralizers | 0.45% |
| Water | qsp 100% |

The above composition may be used as an anti-aging day cream.

### Example 4

### Cosmetic Composition

| | |
|---|---|
| Benzotriazole silicone* | 5% |
| DHEA | 1% |
| Polyglycerol distearate (2 mol) | 2% |
| PEG mono-stearate (8 OE) | 1.35% |
| Stearic acid | 1% |
| Propylene glycol | 10% |
| Preservative | 1.35% |
| Gelling agent | 0.5% |
| Neutralizers | 0.45% |
| Water | qsp 100% |

*2-(2H-benzotriazole-2-yl)-4-methyl-6-[2-methyl-3-[1,3,3,3-tetramethyl-1-[(trimethylsilyl)oxy]disiloxanyl]propynyl]phenol, described in patent application EP-A-0392 883.

This composition may be used to prevent or treat the signs of aging such as wrinkles and lines and cutaneous slackening.

All articles, patents, applications, standards, documents, etc. mentioned above are incorporated herein by reference.

One of ordinary skill in the art is capable of making and using the invention described above and claimed below in view of this disclosure. With regard to use, a person may apply, e.g., 0.1-5g of invention composition to the skin (or other keratin materials such as the hair, lips, nails, scalp, etc.) to be treated once or more per day for one day or several, for example.

UV filters that can specifically be excluded from the invention composition and methods are described above. Other excludable UV filters include aminobenzoic acid, alkylaminobenzoic acid, C₁-C₃ alkyl cinnamates, C₁-C₃ alkoxy, C₆-₉ alkyl cinnamate, and C₁-C₃ alkoxy cinnamate.

Other materials that may be excluded from the invention composition and methods include retinoic acid, and Vitamin A derivatives, especially those discussed in U.S. Pat. No. 6,093,706, incorporated herein by reference (“retinoids”).

1. A composition comprising a DHEA compound and a liposoluble UV filter, provided that said composition does not comprise para-aminobenzoic acid, methyl cinnamate, 4-methoxy octyl cinnamate, titanium oxide or zinc oxide.

2. The composition according to claim 1, comprising one or both of Δ5-pregnenolone and 17α-hydroxy pregnenolone.

3. The composition according to claim 1, comprising one or more of Δ5-androstene-3,17diol, Δ4-androstene-3,17-dione, 7αOH-DHEA, 7βOH-DHEA and 7-keto-DHEA.

4. The composition according to claim 1, comprising 7αOH-DHEA.

5. The composition according to claim 1, comprising from 0.01 to 10% by weight of DHEA compound, with respect to the total weight of the composition.

6. The composition according to claim 5, comprising from 0.01 to 3% by weight of DHEA compound, with respect to the total weight of the composition.

7. The composition according to claim 1, wherein said liposoluble UV filter is selected from the group consisting of:
(1) salicylic acid derivatives;
(2) cinnamic acid derivatives;
(3) liquid β,β′-diphenylacrylate derivatives;
(4) para-aminobenzoic acid derivatives;
(5) dibenzoylmethane derivatives;
(6) liposoluble benzophenone derivatives;
(7) benzotriazole silicones;
(8) silicic derivatives of N-substituted Benzimidazolyl-Benzazoles or of Benzofuranyl-Benzazoles; and
(9) mixtures thereof.

8. The composition according to claim 7, comprising one or both of octyl salicylate and homomenthyl salicylate.

9. The composition according to claim 7, comprising 2-ethylhexyl α-cyano-β,β′diphenylacrylate.

10. The composition according to claim 7, comprising 4-(tert-butyl)4′-methoxy dibenzoylmethane.

11. The composition according to claim 7, comprising 2-(2H-benzotriazole-2-yl)-4-methyl-6-[2-methyl-3-[1,3,3,3-tetramethyl-1-[trimethylsilyl)oxy]disiloxanyl]propynyl]phenol.

12. The composition according to claim 1, comprising from 0.1% to 20% by weight of liposoluble UV filter, with respect to the total weight of the composition.

13. The composition according to claim 12, comprising from 0.5 to 10% by weight of liposoluble UV filter, with respect to the total weight of the composition.

14. The composition according to claim 1, further comprising a hydrosoluble UV filter.

15. The composition according to claim 14, comprising a hydrosoluble UV filter selected from the group consisting of:
(1) filters derived from benzylidene camphor;
(2) benzimidazole or benzoxazole filters;
(3) hydrosoluble derivatives of benzophenone; and
(4) mixtures thereof.

16. The composition according to claim 15, comprising benzene 1,4-[di(3-methylidenecampho 10-sulfonic)] acid.

17. The composition according to claim 15, comprising 2-phenyl benzimidazole 5-sulfonic acid.

18. The composition according to claim 15, comprising 2-hydroxy-4-methoxy-benzophenone-5-sulfonic acid.

19. A composition comprising a DHEA compound and a liposoluble UV filter, provided said composition does not comprise a retinoid .

20. A method to prevent or treat wrinkles and lines and/or cutaneous slackening, comprising topically applying the composition of claim 1 to keratin material of a human.

21. A method to prevent or treat the signs of chronological or actinic cutaneous aging, comprising topically applying the composition claim 1 to keratin material of a human.

22. A process for the solubilization of a DHEA compound, comprising:
mixing said DHEA compound with at least one liposoluble UV filter other than para-aminobenzoic acid, methyl cinnamate, 4-methoxy octyl cinnamate, titanium oxide and zinc oxide.
